(19) United States
(12) Patent Application Publication
(10) Pub. No.: US 2020/0037980 A1
(43) Pub. Date: Feb. 6, 2020

(54) PHOTOGRAPHING SEQUENCE DETERMINATION SUPPORTING METHOD, PHOTOGRAPHING SEQUENCE DETERMINATION SUPPORTING PROGRAM, AND X-RAY RADIOGRAPHIC APPARATUS EQUIPPED THEREWITH

(71) Applicant: Shimadzu Corporation, Kyoto (JP)

(72) Inventor: Shuichi INOMATA, Kyoto (JP)

(21) Appl. No.: 16/605,479

(22) PCT Filed: Apr. 13, 2018
(86) PCT No.: PCT/JP2018/015527

§ 371 Date: Oct. 15, 2019

(30) Foreign Application Priority Data
Apr. 18, 2017 (JP) 2017-081848

(51) Int. Cl. A61B 6/00

(52) U.S. Cl. CPC A61B 6/584; A61B 6/589

(57) ABSTRACT

Elements that determine a photographing sequence include at least a path that minimizes a moving distance or a moving time of a subject, and a path that minimizes a moving distance or a moving time of a component constituting an X-ray radiographic apparatus. In step S1 (select each button), one of the respective elements is selected, and according to the selected element, the photographing sequence is determined in any one of steps S10, S20, and S30. That is, a user selects an element that matches a situation and determines the photographing sequence according to the selected element, thereby allowing the user to freely sort the photographing sequence according to the situation.

### TECHNICAL FIELD

The present invention relates to a photographing sequence determination supporting method, a photographing sequence determination supporting program, and an X-ray radiographic apparatus equipped therewith, and particularly to a technique that supports determination of a photographing sequence in a series of X-ray radiographic processes in association with movement of a subject or the X-ray radiographic apparatus.

### BACKGROUND ART

Regarding the X-ray radiographic apparatus, various conditions for performing fluoroscopy and photographing are not set manually. In general, a series of setting items are preset together in a form such as a “protocol.” Various conditions preset in the photographing protocol include the following items.
X-ray tube (bulb) to be used (for example, focal size)
X-ray detector to be used and a holding part thereof (for example, upright stand, table, free (without a holding part))
X-ray conditions such as tube voltage, tube current, and exposure time of the X-ray tube
Physical conditions such as a distance from a focal point of the X-ray tube to a detection surface of the X-ray detector (source image distance (SID)), angle of the X-ray tube (bulb), grid type, and collimator opening
Image processing conditions after photographing
Position of the X-ray tube (bulb), position of the X-ray detector, and position of the holding part

The photographing protocol is identified with, for example, a protocol name such as “upright, chest, front” and an ID number with the above various conditions as a set.

It should be noted that “photographing” in this specification includes a case where an X-ray image is acquired by emitting an X-ray with a strong dose, and a case where a moving image is displayed by continuously emitting an X-ray with a lower dose and displaying X-ray images successively (perspective).

Before starting an inspection, a user of an X-ray radiographic apparatus (for example, an operator) designates the X-ray radiographic apparatus to use a photographing protocol suitable for obtaining an image necessary for the inspection based on order information or the like issued by the radiology information system (RIS). When photographing is performed a plurality of times under a plurality of conditions, a plurality of photographing protocols is designated. A protocol list is created with the designated protocol group.

The protocol list is created by a method of manually selecting the photographing protocol by the user, a method of automatically selecting the photographing protocol, or the like. Specifically, in the latter method, which photographing protocol is selected for which order from the RIS is mapped in advance, and the photographing protocol is automatically selected in cooperation with the advance mapping at the time of ordering. The protocol list has a sequence, and the photographing protocols are arranged in the sequence of manual selection by the user and in the sequence of the order information from the RIS. Some systems have a function of changing the sequence of the photographing protocols by the user's button operation or mouse operation.

After the inspection is started, the photographing protocols are selected sequentially from the top of the protocol list, and photographing is performed according to each photographing protocol. Some systems have a function of, when photographing is performed according to one photographing protocol, automatically selecting the next photographing protocol in the protocol list.

Photographing according to each photographing protocol (series of X-ray radiographic processes) may involve a change in the X-ray detector (flat panel X-ray detector (FPD), cassette), movement of a subject (patient), movement of the X-ray tube (bulb), and a change in an angle or height of a table (fluoroscopic table). Therefore, the sequence of the photographing protocols to perform is an important factor in efficiently conducting inspection workflow. For example, for an inspection in which a plurality of radiographic processes is performed with each of an upright stand and a table (fluoroscopic table), by arranging the photographing according to the photographing protocol using the upright stand in a first half of the inspection and the photographing according to the photographing protocol using the table in a second half of the inspection, the number of movements of the patient between the upright stand and the table, and the number of movements of system components (photographing system such as the X-ray tube, the X-ray detector, the table) can be reduced to one.

In particular, in a case where the patient has a disease in the foot or is elderly, even in an inspection room, getting on and off the table and moving to the front of the upright stand impose a burden on the patient. Furthermore, a burden is also imposed on an engineer (operator) who supports the patient. In such a case, the user such as an operator manually sorts the sequence of the photographing protocols so as to minimize the moving distance or the moving time of the patient, whereby the inspection can be smoothly advanced.

In fluoroscopy/photography according to each photographing protocol, it is necessary to change a height and angle of the X-ray tube, the X-ray detector, and the table, which are system components, in addition to the patient's position. Positioning of these components needs to be changed for each photographing protocol, and a change operation and change time of the positioning of each component will occur. Moreover, moving a ceiling-suspended X-ray tube (bulb) and a change in a height and angle of the table may cause collision accidents if there is a person or obstacle in a moving path thereof. Therefore, as in a case of a patient, the inspection can be smoothly advanced by the user manually sorting the sequence of the photographing protocols so as to minimize the moving distance or the moving time of these components. At this time, a path with no persons or obstacles is considered.

Meanwhile, there is a system, in a case where there is a plurality of radiation image photographing (FPD), that determines a photographing device in a photographing enabled state and presents to the user an icon of a photographing protocol that can be performed (for example, see Patent Literature 1). The system of Patent Literature 1: WO 2011/142157 A has a function of presenting an appropriate sequence regardless of a sequence of order information.

As described above, the sequence of the photographing protocols to perform is an important factor in efficiently conducting inspection workflow. In particular, if there is a function of, when photographing according to a photographing protocol is performed, automatically selecting a next photographing protocol in the protocol list, the following problems occur. That is, when the original sequence is not appropriate in the photographing protocols, the user needs to manually select the photographing protocol during the inspection, which requires time and effort. Therefore, it is preferable that the sequence of the photographing protocols to perform is set appropriately each time. However, it takes time and effort to change the sequence of the photographing protocols by a button operation or mouse operation for each inspection.

In particular, when the photographing protocol is created in cooperation with the order information from the RIS, the RIS side cannot determine what sequence of the photographing protocols will increase efficiency. As a result, depending on the sequence of order, the sequence of the photographing protocols to perform may be inappropriate. In this case, it takes time and effort for the user who performs the inspection to change the sequence of the photographing protocols.

Patent Literature 1: WO 2011/142157 A makes it possible to discriminate a photographing-ready (that is, performable) photographing protocol from a photographing-not ready photographing protocol by an icon display method. However, it is necessary for the user to select a photographing protocol to perform from discrimination results, and time and effort to select the photographing protocol is not reduced.

Therefore, it is disclosed that, in fields other than X-ray photography, for example, when performing a plurality of inspections such as positron emission tomography (PET) and nuclear magnetic resonance imaging (MRI), the sequence of the photographing protocols that minimizes the time required for image photographing is automatically optimized and generated (for example, see Patent Literature 2). Applying the photographing protocol sequence optimization in Patent Literature 2 to X-ray photography and applying the time required for image photographing in Patent Literature 2 to the moving time make it possible to automatically optimize and generate the sequence of the photographing protocols to minimize the moving time.

### CITATION LIST

### Patent Literature

Patent Literature 1: WO 2011/142157 A

Patent Literature 2: Japanese Patent No. 5288844

### SUMMARY OF INVENTION

### Technical Problem

However, there is a problem that there is a discrepancy regarding the sequence of the photographing protocols between a person who has created the photographing order (for example, a doctor) and an engineer (operator) who actually observes the patient's state and performs X-ray photography. For example, if the engineer (operator) observes the patient's state and determines to perform important photographing first based on observation results, the engineer needs to change the sequence of the photographing protocols from the order information. In this way, there is a desire for the user such as an operator to freely change the sequence of the photographing protocols according to the situation.

The present invention has been made in view of such circumstances, and an object of the present invention is to provide a photographing sequence determination supporting method that allows a user to sort a photographing sequence freely according to a situation, a photographing sequence determination supporting program, and an X-ray radiographic apparatus equipped therewith.

### Solution to Problem

In order to achieve such an object, the present invention has the following configuration.

That is, a photographing sequence determination supporting method according to the present invention is a photographing sequence determination supporting method for supporting determination of a photographing sequence in a series of X-ray radiographic processes in association with movement of a subject or an X-ray radiographic apparatus. The photographing sequence determination supporting method includes: an element selection step of selecting one from elements that determine the photographing sequence, the elements including at least a path that minimizes a moving distance or a moving time of the subject, and a path that minimizes a moving distance or a moving time of a component constituting the X-ray radiographic apparatus; and a photographing sequence determination step of determining the photographing sequence according to a selected element.

### Operation and Effect

According to the photographing sequence determination supporting method according to the present invention, the elements that determine the photographing sequence include at least the path that minimizes a moving distance or a moving time of the subject, and the path that minimizes a moving distance or a moving time of the component constituting the X-ray radiographic apparatus. One is selected from the respective elements, and the photographing sequence is determined according to the selected element. That is, a user selects an element that matches a situation and determines the photographing sequence according to the selected element, thereby allowing the user to freely sort the photographing sequence according to the situation.

When the user selects the path that minimizes the moving distance or the moving time of the subject, the photographing sequence along such a path is determined. When the user selects the path that minimizes the moving distance or the moving time of the component constituting the X-ray radiographic apparatus, the photographing sequence along such a path is determined. In either case of the subject or the component, by allowing the user to select the path that minimizes the moving distance or the moving time and determining the photographing sequence along the selected path, the photographing sequence can be optimized automatically.

The photographing sequence determination supporting method according to the present invention preferably includes: a position input step of inputting a position of the component; and a photographing sequence update step of performing update to sort the photographing sequence with the input position of the component as a starting point. When the user selects the path that minimizes the moving distance or the moving time of the component, the shortest path changes with the current position of the component as a starting point. Therefore, by performing the position input step and the photographing sequence update step, the update to sort the photographing sequence can be automatically performed with the input position of the component as a starting point.

Unless the component is moved between the previous inspection and the current inspection, the final position of the component in the previous inspection matches the current position of the component in the current inspection. Anyway, the final position of the component in the previous inspection may be input, or the current position of the component in the current inspection may be input. An input unit to be used in the position input step is not limited to a pointing device or a button manually input by the user, but may be an input port. When the input unit is the input port, a position detector that detects the position of the component is provided, and the position of the component detected by the position detector is transmitted to the input port and input.

In either case of the subject or the component, an algorithm to be used for searching for the path that minimizes the moving distance or the moving time is not particularly limited. For example, it is possible to use an algorithm such as a method of searching for the shortest path when all of a plurality of points A, B, . . . are visited like a picture drawn with one stroke (generally referred to as “traveling salesperson problem”).

In either case of the subject or the component, the photographing sequence determination supporting method according to the present invention preferably includes: a cost correction step of correcting a cost including the moving distance or the moving time in part of the path. Changing the moving distance or the moving time in part of the path without changing the path itself means “correcting the cost (distance or time).” For example, in a case where the subject (patient) has a disease in the foot as described above, when photographing that includes getting on and off a table is included in a series of X-ray radiographic processes, even if the moving distance or the moving time is short, correction is made to increase the cost if getting on and off a table is included. In this way, the path that minimizes the moving distance or the moving time is searched for, taking into account the cost corrected according to the situation.

Another example of the elements other than the path that minimizes the moving distance or the moving time is preset setting values. The photographing sequence is determined in the photographing sequence determination step in ascending order or descending order of the setting values. By using one of the various setting values (setting parameters) in photographing, such as the X-ray tube (bulb) to use or the X-ray detector to use, the photographing sequence is determined in ascending/descending order. For example, when there is a plurality of X-ray tubes or there is a plurality of X-ray detectors, the priority to use is set as the setting value, and photographing regarding the X-ray tube/X-ray detector with high priority is first performed sequentially.

For example, a case will be described where in a system in which two “X-ray detectors to use” use battery-powered wireless FPDs, the battery of one “FPD 1” is dead and the power is not turned on. The setting value indicating priority of the other “FPD 2” is set at “1”, and the setting value indicating priority of “FPD 1” with the dead battery and the power turned off is set at “2.” It is possible to make settings to postpone the photographing using “FPD 1” in order to charge “FPD 1” and perform the inspection first by photographing using “FPD 2.” Remaining power of each wireless FPD may be monitored, and monitoring results (remaining power) may be converted into a numerical form and set as a setting value indicating priority. If the element is a preset setting value, an effect of being able to perform photographing without changing the photographing sequence of desired minimum photographing, regardless of other elements, can be also produced.

The photographing sequence determination supporting program according to the present invention causes a computer to execute the photographing sequence determination supporting method according to the present invention.

### Operation and Effect

The photographing sequence determination supporting program according to the present invention causes a computer to execute the photographing sequence determination supporting method according to the present invention, whereby a user can freely sort the photographing sequence according to the situation.

An X-ray radiographic apparatus according to the present invention is an X-ray radiographic apparatus including the photographing sequence determination supporting program according to the present invention, the X-ray radiographic apparatus including an arithmetic unit configured to execute the photographing sequence determination supporting program.

### Operation and Effect

The X-ray radiographic apparatus according to the present invention includes the arithmetic unit configured to execute the photographing sequence determination supporting program according to the present invention, whereby a user can freely sort the photographing sequence according to the situation.

### Advantageous Effects of Invention

According to the photographing sequence determination supporting method, the photographing sequence determination supporting program, and the X-ray radiographic apparatus equipped therewith according to the present invention, the elements that determine the photographing sequence include at least the path that minimizes the moving distance or the moving time of the subject, and the path that minimizes the moving distance or the moving time of the component constituting the X-ray radiographic apparatus. One is selected from the respective elements, and the photographing sequence is determined according to the selected element. That is, a user selects an element that matches a situation and determines the photographing sequence according to the selected element, thereby allowing the user to freely sort the photographing sequence according to the situation.

When the user selects the path that minimizes the moving distance or the moving time of the subject, the photographing sequence along such a path is determined. When the user selects the path that minimizes the moving distance or the moving time of the component constituting the X-ray radiographic apparatus, the photographing sequence along such a path is determined. In either case of the subject or the component, by allowing the user to select the path that minimizes the moving distance or the moving time and determining the photographing sequence along the selected path, the photographing sequence can be optimized automatically.

### BRIEF DESCRIPTION OF DRAWINGS

FIG. 1 is a schematic perspective view of an X-ray radiographic apparatus according to an embodiment.

FIG. 2 is a block diagram of the X-ray radiographic apparatus according to the embodiment according to the embodiment.

FIG. 3 is a flowchart of photographing sequence determination support according to the embodiment.

FIG. 4 is an implementation aspect of a protocol editing screen regarding photographing sequence (photographing protocol sequence).

FIG. 5 is an implementation aspect of a moving cost correction screen.

FIG. 6 is a schematic plan view in which layout of an inspection room and a movable range of an X-ray tube (second bulb) of a ceiling traveling unit in a sequence in which a moving cost of a subject (patient) is the shortest are shown together.

FIG. 7 is a schematic plan view in which layout of the inspection room and the movable range of the X-ray tube (second bulb) of the ceiling traveling unit in which a moving cost of components constituting the X-ray radiographic apparatus is the shortest are shown together.

FIG. 8 is a schematic view for describing skyline photography.

FIGS. 9 (a) and (b) are display aspects of a protocol list that has been sorted.

### DESCRIPTION OF EMBODIMENTS

An embodiment of the present invention will be described below with reference to the drawings.

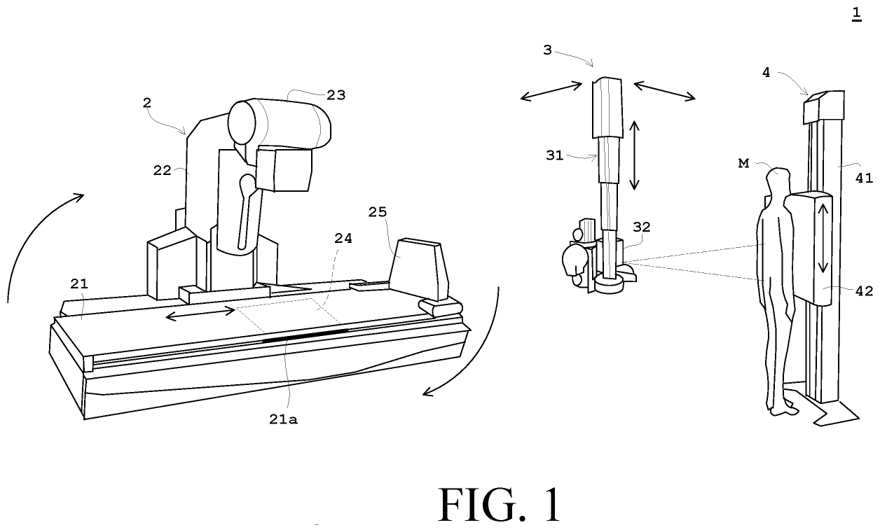

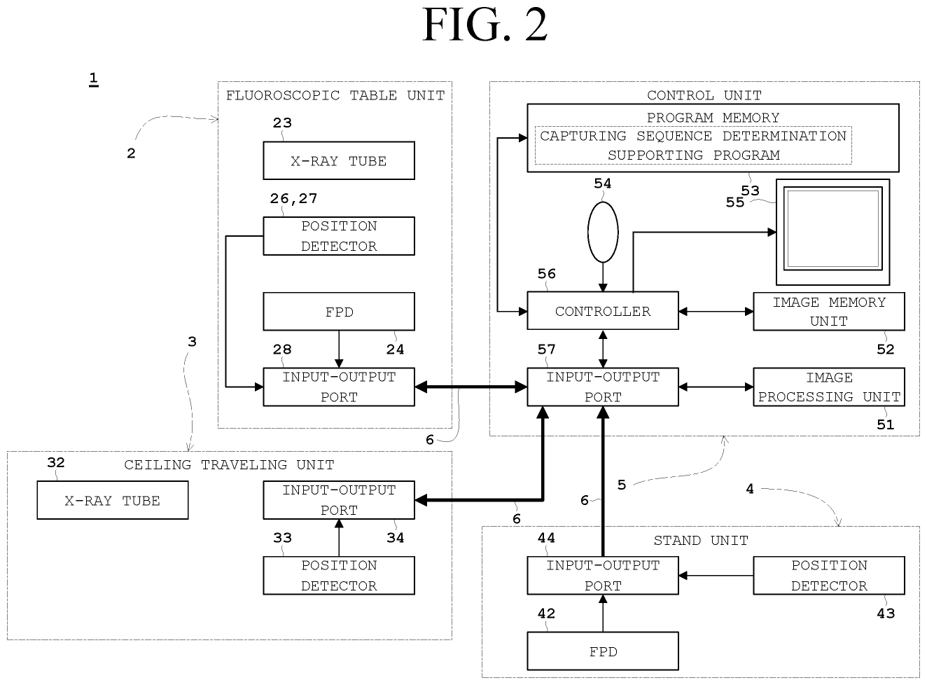

FIG. 1 is a schematic perspective view of an X-ray radiographic apparatus according to the embodiment, and FIG. 2 is a block diagram of the X-ray radiographic apparatus according to the embodiment. In the present embodiment, a flat panel X-ray detector (FPD: flat panel detector) will be described as an example of an X-ray detection unit.

As shown in FIG. 1, the X-ray radiographic apparatus 1 according to the present embodiment includes: a fluoroscopic table unit 2 that performs X-ray photography by using a fluoroscopic table 21 that can recline (incline) a subject M in a horizontal posture (decubitus posture), an inclined posture, or an upright posture; a ceiling traveling unit 3 that suspends and supports an X-ray tube 32 such that the X-ray tube 32 can move (can travel) along the ceiling and emits an X-ray from the X-ray tube 32; a stand unit 4 that performs X-ray photography with the subject M in an upright posture; and a control unit 5 (not shown in FIG. 1) that performs image processing on a subject M's X-ray image and performs photographing sequence determination support described later.

As shown in FIG. 2, the fluoroscopic table unit 2, the ceiling traveling unit 3, the stand unit 4, and the control unit 5 are electrically connected to one another with communication cables 6. With the communication cables 6, the fluoroscopic table unit 2, the ceiling traveling unit 3, the stand unit 4, and the control unit 5 are communicatively configured to one another.

As shown in FIG. 1, the fluoroscopic table unit 2 includes: the fluoroscopic table 21 that can recline (incline) the subject M in a horizontal posture (decubitus posture), an inclined posture, or an upright posture; a strut 22 that can move along a longitudinal direction of the fluoroscopic table 21 (longitudinal direction of the subject M); an X-ray tube 23 and a flat panel X-ray detector (FPD) 24 supported by the strut 22; and a footrest 25 for supporting the subject M in an upright posture or an inclined posture. Also, as shown in FIG. 2, the fluoroscopic table unit 2 includes a position detector 26 that detects a position of the X-ray tube 23, a position detector 27 that detects a position of the FPD 24, and an input-output port 28. Note that in a similar manner to the control unit 5, the fluoroscopic table unit 2 may include an input unit, an output unit, and a controller.

As shown in FIG. 1, the ceiling traveling unit 3 includes a strut 31 that can move (can travel) along the ceiling, and the X-ray tube 32 that is supported by the strut 31 and can be adjusted in an orientation. Also, as shown in FIG. 2, the ceiling traveling unit 3 includes a position detector 33 that detects a position and angle of the X-ray tube 32 and an input-output port 34. Note that in a similar manner to the control unit 5, the ceiling traveling unit 3 may include an input unit, an output unit, and a controller.

As shown in FIG. 1, the stand unit 4 includes an upright stand 41 that supports the subject M in an upright posture, and a flat panel X-ray detector (FPD) 42 that is mounted on the upright stand 41 and can be moved up and down. Also, as shown in FIG. 2, the stand unit 4 includes a position detector 43 that detects a position of the FPD 42 and an input-output port 44. Note that in a similar manner to the control unit 5, the stand unit 4 may include an input unit, an output unit, and a controller.

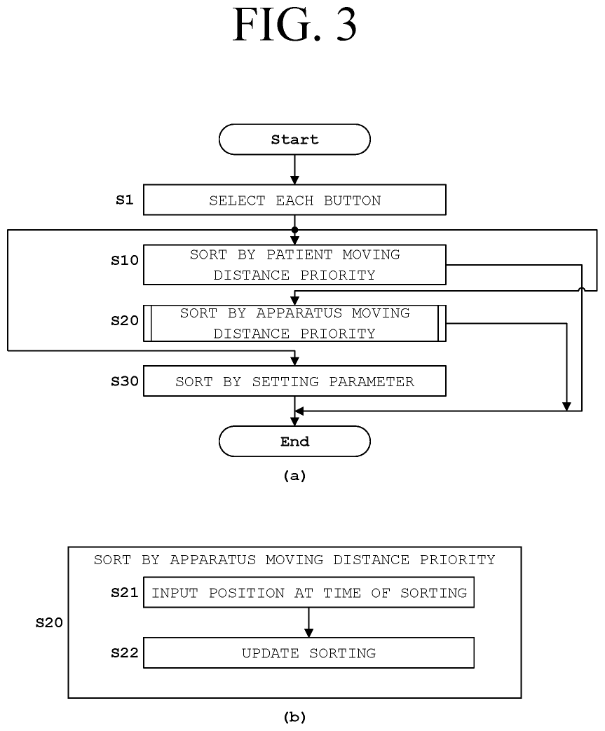

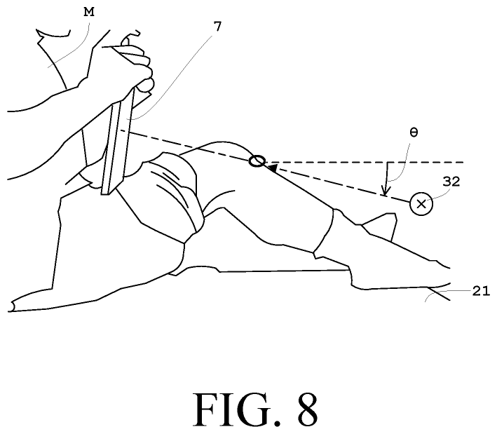

As shown in FIG. 2, the control unit 5 includes an image processing unit 51 that performs image processing on an X-ray image obtained by the FPD 24 of the fluoroscopic table unit 2, the FPD 42 of the stand unit 4, or a portable wireless FPD (for example, see an FPD 7 used for skyline photography shown in FIG. 8). In addition, the control unit 5 includes: an image memory unit 52 that writes and stores the X-ray image obtained by the FPD 24, the FPD 42, or the wireless FPD, and an X-ray image that undergoes image processing by the image processing unit 51; and a program memory 53 that stores in advance the photographing sequence determination supporting program described later. Furthermore, the control unit 5 includes an input unit 54, an output unit 55, a controller 56, and an input-output port 57. The specific photographing sequence determination supporting program will be described later with reference to FIGS. 3 to 9 and Tables 1 to 17. The controller 56 corresponds to an arithmetic unit in the present invention.

As shown in FIG. 1, the fluoroscopic table 21 of the fluoroscopic table unit 2 can incline the subject M into a horizontal posture (decubitus posture), an inclined posture, or an upright posture. In conjunction with the inclination of the fluoroscopic table 21, the strut 22 is inclined, and the X-ray tube 23 and the FPD 24 supported by the strut 22 are also inclined.

The subject M lies in a horizontal posture on the fluoroscopic table 21 in a horizontal state. After the subject M in a horizontal posture brings the foot into contact with the footrest 25, the subject M is supported in an upright posture or an inclined posture by inclining the fluoroscopic table 21. Supporting the subject M in this way enables X-ray photography of the subject M in an upright posture or an inclined posture. Of course, the subject M in an upright posture may be supported by the subject M in an upright posture placing the foot on the footrest 25 in the fluoroscopic table 21 in a standing state and the subject M in an upright posture leaning against the fluoroscopic table 21 in a standing state. Furthermore, the subject M in an inclined posture may be supported by inclining the fluoroscopic table 21 from a state in which the subject M in an upright posture is supported.

In addition, the fluoroscopic table 21 is provided with an insertion port 21a, and the FPD 24 of the fluoroscopic table unit 2 is loaded directly under a mounting surface of the fluoroscopic table 21 through the insertion port 21a. The strut 22 of the fluoroscopic table unit 2 is configured to move along a longitudinal direction of the fluoroscopic table 21 (longitudinal direction of the subject M). In conjunction with the longitudinal movement of the strut 22, the X-ray tube 23 and the FPD 24 (of the fluoroscopic table unit 2) supported by the strut 22 move in a longitudinal direction while maintaining a state of facing each other. The longitudinal movement of the X-ray tube 23 and the FPD 24 enables X-ray photography of the subject M at a desired position. Also, this enables photographing of a long image larger than a size of the FPD 24 in a longitudinal direction.

As shown in FIG. 2, the position detector 26 is disposed for the X-ray tube 23, and the position detector 26 detects a position of the X-ray tube 23. Similarly, the position detector 27 is disposed for the FPD 24, and the position detector 27 detects a position of the FPD 24. The position detectors 26 and 27 include, for example, potentiometers. The positions of the X-ray tube 23 and the FPD 24 detected by the position detectors 26 and 27 are transferred to the input-output port 57 of the control unit 5 via the input-output port 28 and the communication cable 6.

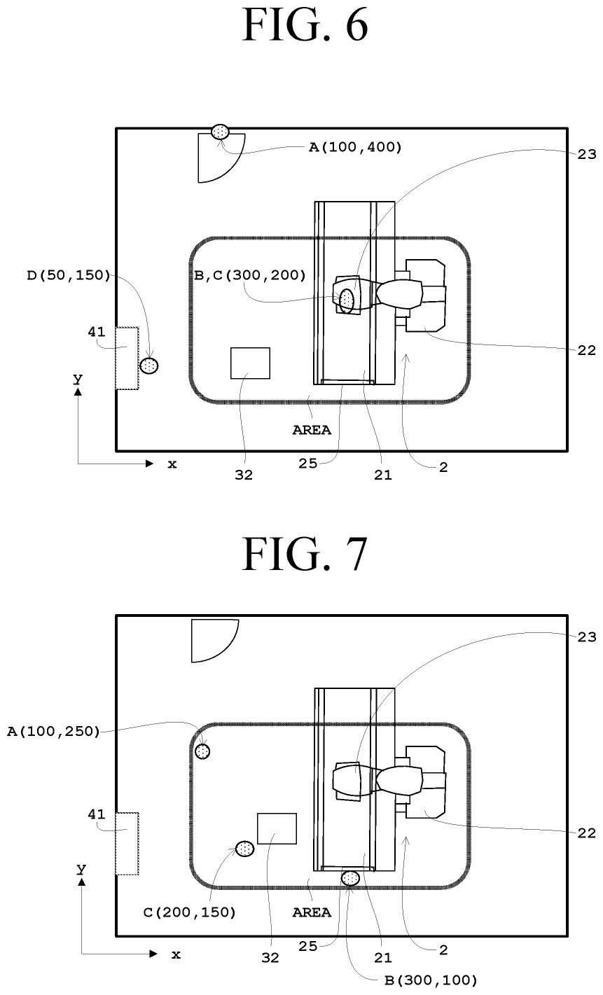

As shown in FIG. 1, the strut 31 of the ceiling traveling unit 3 can move (can travel) along a rail (not shown) laid along the ceiling. The rail is laid along x and y directions shown in FIGS. 6 and 7 (along the ceiling), and the strut 31 can move (can travel) along the x and y directions. The strut 31 is configured to expand and contract, and the strut 31 supports the X-ray tube 32 of the ceiling traveling unit 3, thereby allowing the X-ray tube 32 to move horizontally/up and down. Also, an orientation of the X-ray tube 32 can be adjusted. Therefore, the X-ray photography in an upright posture is possible by moving the X-ray tube 32 horizontally/up and down and adjusting the orientation toward the upright stand 41 of the stand unit 4. Furthermore, X-ray photography other than the X-ray photography with the fluoroscopic table unit 2 is possible (for example, skyline photography shown in FIG. 8, tomosynthesis obtained from an oblique direction).

As shown in FIG. 2, the position detector 33 is disposed for the X-ray tube 32, and the position detector 33 detects a position and angle of the X-ray tube 32. In a similar manner to the position detectors 26 and 27 of the fluoroscopic table unit 2, the position detector 33 also includes a potentiometer. The position and angle of the X-ray tube 32 detected by the position detector 33 are transferred to the input-output port 57 of the control unit 5 via the input-output port 34 and the communication cable 6.

As shown in FIG. 1, the upright stand 41 of the stand unit 4 is installed with respect to the floor surface. The FPD 42 of the stand unit 4 can move up and down along the upright stand 41.

As shown in FIG. 2, the position detector 43 is disposed for the FPD 42, and the position detector 43 detects a position of the FPD 42. In a similar manner to the position detectors 26 and 27 of the fluoroscopic table unit 2, the position detector 43 also includes a potentiometer. The position of the FPD 42 detected by the position detector 43 is transferred to the input-output port 57 of the control unit 5 via the input-output port 44 and the communication cable 6.

The image processing unit 51 of the control unit 5 and the controller 56 of the control unit 5 include central processing units (CPU) or the like. Note that the image processing unit 51 may include a graphics processing unit (GPU) or the like.

The image memory unit 52 of the control unit 5 writes, stores, and reads as necessary the X-ray image obtained by the FPD 24 of the fluoroscopic table unit 2, the FPD 42 of the stand unit 4, or the wireless FPD, and the X-ray image that undergoes image processing by the image processing unit 51. The photographing sequence determination supporting program is stored in advance in the program memory 53 of the control unit 5. The photographing sequence determination supporting program is read from the program memory 53 to the controller 56, and the photographing sequence determination supporting program is executed by the controller 56, thereby performing the photographing sequence determination support shown in the flowchart of FIG. 3. The image memory unit 52 includes a storage medium represented by a random-access memory (RAM) or the like, and the program memory 53 includes a storage medium represented by a read-only memory (ROM).

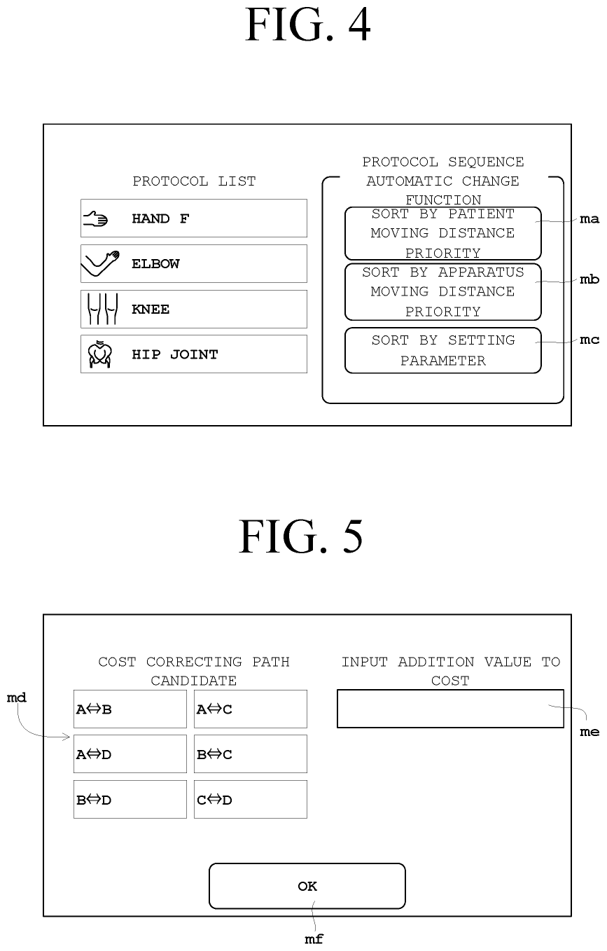

The input unit 54 of the control unit 5 transfers data and commands input by a user such as an operator to the controller 56. The input unit 54 includes a pointing device represented by a mouse, a keyboard, a joystick, a trackball, a touch panel, or the like. In the present embodiment, one of a plurality of buttons (three buttons ma to mc in FIG. 4) in a “protocol sequence automatic change function” shown in FIG. 4 is selected by an input operation into the input unit 54 (for example, a mouse click operation).

The output unit 55 of the control unit 5 includes a display unit represented by a monitor, a printer, or the like. When the output unit 55 is a display unit, the output is displayed, and when the output unit 55 is a printer, the output is printed. In the present embodiment, the display unit outputs and displays a protocol editing screen shown in FIG. 4 or a moving cost correction screen shown in FIG. 5.

The controller 56 of the control unit 5 comprehensively controls each part constituting the control unit 5. The fluoroscopic table unit 2, the ceiling traveling unit 3, the stand unit 4, and the control unit 5 are electrically connected with the communication cables 6. Through such connection, the fluoroscopic table unit 2, the ceiling traveling unit 3, the stand unit 4, and the control unit 5 are communicatively configured to one another. Therefore, the controller 56 can comprehensively control each part constituting the fluoroscopic table unit 2, the ceiling traveling unit 3, and the stand unit 4 via the communication cables 6.

For example, the controller 56 drives and controls the fluoroscopic table 21 and the strut 22 (see FIG. 1 for both) of the fluoroscopic table unit 2, the strut 31 of the ceiling traveling unit 3 (see FIG. 1), and the upright stand 41 of the stand unit 4 (see FIG. 1). That is, the controller 56 controls motors (not shown), thereby driving the fluoroscopic table 21, the struts 22 and 31, and the upright stand 41 by the motors. By driving by the motors, in conjunction with the driving of the fluoroscopic table 21, the struts 22 and 31, and the upright stand 41, it is possible to control the X-ray tube 23 and the FPD 24 of the fluoroscopic table unit 2, the X-ray tube 32 of the ceiling traveling unit, and the FPD 42 of the stand unit 4 to move to desired positions.

The X-ray photography in an upright posture includes a method of performing X-ray photography by the fluoroscopic table unit 2 and a method of performing X-ray photography by the ceiling traveling unit 3 and the stand unit 4.

When the X-ray photography is performed by the fluoroscopic table unit 2, the fluoroscopic table 21 of the fluoroscopic table unit 2 is set in a standing state, and the X-ray tube 23 of the fluoroscopic table unit 2 emits an X-ray horizontally. Specifically, the controller 56 drives and controls the fluoroscopic table 21 to be inclined such that the fluoroscopic table 21 is in a standing posture. When the fluoroscopic table 21 becomes in a standing posture, the controller 56 drives and controls the strut 22 of the fluoroscopic table unit 2 such that the X-ray tube 23 and the FPD 24 of the fluoroscopic table unit 2 are positioned at desired positions. When the X-ray tube 23 and the FPD 24 are at desired positions, the controller 56 controls the X-ray tube 23 so as to emit an X-ray toward the subject M in an upright posture (see FIG. 1). The FPD 24 detects the X-ray that has passed through the subject M, thereby obtaining an X-ray image. The X-ray image obtained by the FPD 24 is transferred to the input-output port 57 of the control unit 5 via the input-output port 28 and the communication cable 6. Then, the image processing unit 51 performs image processing on the X-ray image.

When the X-ray photography is performed by the ceiling traveling unit 3 and the stand unit 4, the orientation of the X-ray tube 32 is adjusted such that the X-ray tube 32 of the ceiling traveling unit 3 emits an X-ray to the upright stand 41 of the stand unit 4. Specifically, the controller 56 drives and controls the strut 31 of the ceiling traveling unit 3 such that the X-ray tube 32 is positioned at a desired position and the X-ray tube 32 faces in a desired direction (not a horizontal direction but an oblique direction in a case of tomosynthesis). Meanwhile, the controller 56 drives and controls the upright stand 41 such that the FPD 42 of the stand unit 4 is positioned at a desired position. When the X-ray tube 32 is positioned at a desired position and the X-ray tube 32 faces in a desired direction and the FPD 42 is positioned at a desired position, the controller 56 controls the X-ray tube 32 to emit an X-ray toward the subject M in an upright posture. The FPD 42 detects the X-ray that has passed through the subject M, thereby obtaining an X-ray image. The X-ray image obtained by the FPD 42 is transferred to the input-output port 57 of the control unit 5 via the input-output port 44 and the communication cable 6. Then, the image processing unit 51 performs image processing on the X-ray image.

In the X-ray photography in a horizontal posture, the controller 56 drives and controls the fluoroscopic table 21 to be inclined such that the fluoroscopic table 21 of the fluoroscopic table unit 2 is in a horizontal posture. When the fluoroscopic table 21 is in a horizontal posture, the controller 56 drives and controls the strut 22 of the fluoroscopic table unit 2 such that the X-ray tube 23 and the FPD 24 of the fluoroscopic table unit 2 are positioned at desired positions. When the X-ray tube 23 and the FPD 24 are positioned at desired positions, the controller 56 controls the X-ray tube 23 to emit an X-ray toward the subject M in a horizontal posture. The FPD 24 detects the X-ray that has passed through the subject M, thereby obtaining an X-ray image. The X-ray image obtained by the FPD 24 is transferred to the input-output port 57 of the control unit 5 via the input-output port 28 and the communication cable 6. Then, the image processing unit 51 performs image processing on the X-ray image.

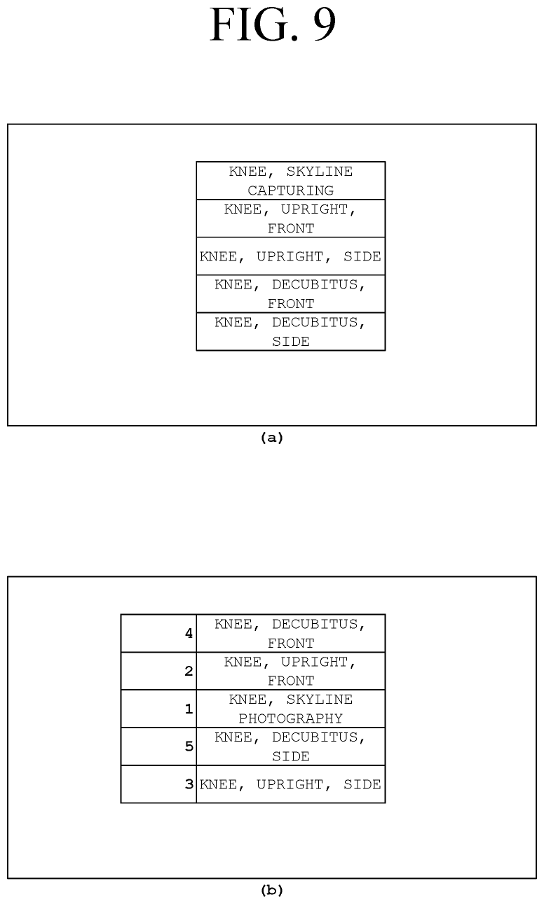

Next, the specific photographing sequence determination supporting program will be described with reference to FIGS. 3 to 9. FIG. 3 is a flowchart of photographing sequence determination support according to the embodiment. FIG. 4 is an implementation aspect of the protocol editing screen regarding photographing sequence (photographing protocol sequence). FIG. 5 is an implementation aspect of the moving cost correction screen. FIG. 6 is a schematic plan view in which layout of an inspection room and a movable range of the X-ray tube (second bulb) of the ceiling traveling unit in a sequence in which the moving cost of the subject (patient) is the shortest are shown together. FIG. 7 is a schematic plan view in which layout of the inspection room and the movable range of the X-ray tube (second bulb) of the ceiling traveling unit in which the moving cost of components constituting the X-ray radiographic apparatus is the shortest are shown together. FIG. 8 is a schematic view for describing skyline photography. FIGS. 9 (a) and 9 (b) are display aspects of a protocol list that has been sorted.

In the following description, the X-ray tube 23 of the fluoroscopic table unit 2 (see FIGS. 1 and 2 for both) is referred to as a “first bulb”, and the X-ray tube 32 of the ceiling traveling unit 3 (see FIGS. 1 and 2 for both) is referred to as a “second bulb.” First, the photographing sequence determination supporting program is read from the program memory 53 of the control unit 5 (see FIG. 2) to the controller 56 of the control unit 5 (see FIG. 2), and the controller 56 executes the photographing sequence determination supporting program. Then, the display unit of the output unit 55 of the control unit 5 (see FIG. 2) outputs and displays the protocol editing screen as shown in FIG. 4. Then, according to the protocol editing screen, the photographing sequence determination support shown in the flowchart of FIG. 3 is performed.

### (Step S1) Select Each Button

As shown in FIG. 4, in the protocol editing screen, for example, a protocol list is displayed on the left side of the screen and a “protocol sequence automatic change function” is displayed on the right side of the screen. A plurality of buttons is displayed in the “protocol sequence automatic change function.” The user such as an operator selects one of the plurality of buttons by an input operation (for example, a mouse click operation) to the input unit 54 of the control unit 5 (see FIG. 2). In FIG. 4, the three buttons are ma to mc, and one button is selected from among the “sort by patient moving distance priority” button ma, the “sort by apparatus moving distance priority” button mb, and the “sort by setting parameter” button mc.

In this way, FIG. 4 displays with the buttons elements that determine the photographing sequence. Details of each button are not particularly limited, and include at least a path that minimizes the moving distance or the moving time of the subject and a path that minimizes the moving distance or the moving time of the component constituting the X-ray radiographic apparatus. In FIG. 4, the “sort by patient moving distance priority” button ma corresponds to the path that minimizes the moving distance or the moving time of the subject, and the “sort by apparatus moving distance priority” button mb corresponds to the path that minimizes the moving distance or the moving time of the component constituting the X-ray radiographic apparatus.

When the “sort by patient moving distance priority” button ma is selected, the process proceeds to step S10. When the “sort by apparatus moving distance priority” button mb is selected, the process proceeds to step S20. When the “sort by setting parameter” button mc is selected, the process proceeds to step S30. Note that in either case of the subject or the component (in FIG. 4, when either one of the “sort by patient moving distance priority” button ma or the “sort by apparatus moving distance priority” button mb is selected), in the path that minimizes the moving distance or the moving time, it is necessary to sort the photographing sequence and perform all the photographing protocols in the protocol list.

When performing all the photographing protocols in the protocol list by sorting the photographing sequence, it is preferable to use an algorithm such as a method of searching for the shortest path when all of a plurality of points A, B, . . . are visited like a picture drawn with one stroke (for example, traveling salesperson problem). When N is the number of points to visit, there will be N. (N factorial) paths. In this specification, the number of N is 3 for simple description. Thus, when the number of N is small, all the combinations may be calculated as follows. When the number of N is large, the algorithm that solves the traveling salesperson problem described above can be used. This step S1 corresponds to the element selection step in the present invention.

### (Step S10) Sort by Patient Moving Distance Priority

When the “sort by patient moving distance priority” button ma is selected, the photographing sequence is determined as follows. It is assumed that the protocol list as shown in Table 1 has been created as an initial state.

TABLE 1

| No | Protocol name | Bulb | Position of patient |
|---|---|---|---|
| 1 | Knee, decubitus, front | First | B Fluoroscopic |
| | | | table (horizontal) |
| 2 | Knee, upright, front | Second | D Stand |
| 3 | Entire lower limbs, long, | First | C Fluoroscopic |
| | upright, front | | table (reclined) |
| 4 | Knee, decubitus, side | First | B Fluoroscopic |
| | | | table (horizontal) |
| 5 | Knee, upright, side | Second | D Stand |

In order to simplify the calculation of the patient's position including a doorway, each position is expressed on two-dimensional coordinates when the inspection room is viewed from above as shown in FIG. 6. Each position is determined when the apparatus is installed. Table 2 summarizes a relationship of coordinates at each position. Note that regarding the positions B and C, as shown in FIG. 6, the fluoroscopic table 21 of the fluoroscopic table unit 2 (see also FIG. 1) is simply horizontal or reclined, and the position of the subject (patient) does not change. A range AREA surrounded by a thick frame in FIG. 6 is a movable range of the X-ray tube 32 (second bulb) of the ceiling traveling unit 3.

TABLE 2

| | Position of patient | x | y |
|---|---|---|---|
| A | Doorway | 100 | 400 |
| B | Fluoroscopic table | 300 | 200 |
| | (horizontal) | | |
| C | Fluoroscopic table | 300 | 200 |
| | (reclined) | | |
| D | Stand | 50 | 150 |

At this time, the distances between respective positions can be calculated as shown in Table 3. In order to simplify the calculation, the moving cost is calculated by rounding the distance to the first digit.

TABLE 3

| Path | Distance | Cost |
|---|---|---|
| A<=>B | 282.8427125 | 280 |
| A<=>C | 282.8427125 | 280 |
| A<=>D | 254.9509757 | 250 |
| B<=>C | 0 | 0 |
| B<=>D | 254.9509757 | 250 |
| C<=>D | 254.9509757 | 250 |

It is assumed that the patient always starts moving from an “A doorway” and finally arrives at the “A doorway.” The total moving cost of the protocol list in the initial state as shown in Table 1 is as shown in Table 4.

TABLE 4

| | | Total moving |
|---|---|---|
| Path | Calculation of moving cost | cost |
| ABDCBDA | “280 + 250 + 250 + 0 + 250 + 250” | 1280 |

A sequence of the protocol list that minimizes the total moving cost is considered. First, it is assumed that the protocols with the same patient position are always performed continuously. In this example, it is assumed that the protocols at the position B of the protocol number 1 and the protocol number 4, and the protocols at the position D of the protocol number 2 and the protocol number 5 in Table 1 are always performed continuously. Possible combinations are 3. (3 factorial)=6 ways, which is the number of sorting at 3 locations B, C, and D, excluding the “A doorway.” Table 5 shows the total moving cost for all the combinations.

TABLE 5

| | | Total moving |
|---|---|---|
| Path | Calculation of moving cost | cost |
| ABCDA | “280 + 0 + 250 + 250” | 780 |
| ABDCA | “280 + 250 + 250 + 280” | 1060 |
| ACBDA | “280 + 0 + 250 + 250” | 780 |
| ACDBA | “280 + 250 + 250 + 280” | 1060 |
| ADBCA | “250 + 250 + 0 + 280” | 780 |
| ADCBA | “250 + 250 + 0 + 280” | 780 |

From Table 5, one of the paths with the smallest total moving cost is “ABCDA”, and the protocol list that is sorted in this sequence is as shown in Table 6. It should be noted from Table 5 that there are “ACBDA”, “ADBCA”, and “ADCBA” in addition to “ABCDA” as the paths with the smallest total moving cost.

TABLE 6

| No | Protocol name | Bulb | Position of patient |
|---|---|---|---|
| 1 | Knee, decubitus, front | First | B Fluoroscopic |
| | | | table (horizontal) |
| 4 | Knee, decubitus, side | First | B Fluoroscopic |
| | | | table (horizontal) |
| 3 | Entire lower limbs, long, | First | C Fluoroscopic |
| | upright, front | | table (reclined) |
| 2 | Knee, upright, front | Second | D Stand |
| 5 | Knee, upright, side | Second | D Stand |

Note that when the moving cost is to be corrected, the moving cost correction screen as shown in FIG. 5 is output and displayed. The reason for correcting the moving cost is described. Even if the moving distance is short, the ease of the patient getting on and off the fluoroscopic table differs between the fluoroscopic table in a horizontal state and the fluoroscopic table in a standing state. There is a circumstance that getting on the fluoroscopic table in a horizontal state is a burden on elderly patients and patients with leg diseases, just as getting on a high bed. In addition, as shown in FIG. 6, there is a case where a device that is an obstacle for the patient is placed between the doorway and the upright stand 41 of the stand unit 4 (see also FIG. 1). Therefore, there is a circumstance in which it is preferable to avoid moving between the “A doorway” and the “D stand” as much as possible.

Therefore, in order to implement sorting the list that takes the above circumstances into consideration, the moving cost is appropriately corrected as shown in Table 7. Then, it is required at least to select the path that minimizes the total moving cost. In Table 7, 100 is added to the moving cost including the “B fluoroscopic table (horizontal)”, and 50 is added to the moving cost between the “A doorway” and the “D stand.”

TABLE 7

| | Cost before | Cost after |
|---|---|---|
| Path | correction | correction |
| A<=>B | 280 | 380 |
| A<=>C | 280 | 280 |
| A<=>D | 250 | 300 |
| B<=>C | 0 | 0 |
| B<=>D | 250 | 350 |
| C<=>D | 250 | 250 |

As shown in FIG. 5, in the moving cost correction screen, for example, “cost correcting path candidate” buttons md are displayed on the left side of the screen. From the “cost correcting path candidate” buttons md, the user such as an operator selects a path of which the cost is to be corrected by an input operation (for example, a mouse click operation) to the input unit 54 of the control unit 5 (see FIG. 2).

In the case of Table 7, there are three paths of which the cost is to be corrected: “A<=>B”, “A<=>D”, and “B<=>D”. Of those paths, in order to input an addition value “100” to the moving cost including the “B fluoroscopic table (horizontal)”, “A<=>B” and “B<=>D” are selected from the “cost correcting path candidate” buttons md, and “100” is input in a “input addition value to cost” field me displayed on the upper right side of the moving cost correction screen shown in FIG. 5. Meanwhile, in order to input the addition value “50” to the moving cost between the “A doorway” and the “D stand”, “A<=>D” is selected from the “cost correcting path candidate” buttons md, and “50” is input in the “input addition value to cost” field me. Then, in order to correct the cost, the “OK” button mf is pressed by, for example, a mouse click operation.

Note that in “B<=>C”, the fluoroscopic table is changed only between horizontal and reclined, and thus the moving cost is “0” and there is no need to correct the cost. In FIG. 5, the addition value is added to correct the cost, but the cost may be corrected by multiplying the moving cost by a weighting factor, for example.

The cost correction described above is not particularly limited if the cost correction is performed before the sorting. In order to correct the cost before step S1 (select each button), after correcting the moving cost on the moving cost correction screen shown in FIG. 5, the button may be selected on the protocol editing screen shown in FIG. 4. Alternatively, in order to correct the cost after step S1 (select each button), after the button is selected on the protocol editing screen shown in FIG. 4, the moving cost may be corrected on the moving cost correction screen shown in FIG. 5.

### (Step S20) Sort by Apparatus Moving Distance Priority

When the “sort by apparatus moving distance priority” button mb is selected, the photographing sequence is determined as follows. Here, in order to simplify the description, as the system component, only the moving cost of the X-ray tube 32 (second bulb) of the ceiling traveling unit 3 will be described. It is assumed that the protocol list as shown in Table 8 has been created as the initial state.

TABLE 8

| | | | Position of | Position of |
|---|---|---|---|---|
| No | Protocol name | Bulb | patient | second bulb |
| 1 | Knee, decubitus, | First | Fluoroscopic | A Evacuation |
| | front | | table | position |
| | | | (horizontal) | |
| 2 | Knee, upright, | Second | Stand | C For stand |
| | front | | | |
| 3 | Knee, skyline | Second | Fluoroscopic | B For skyline |
| | photography | | table | |
| | | | (horizontal) | |
| 4 | Knee, decubitus, | First | Fluoroscopic | A Evacuation |
| | side | | table | position |
| | | | (horizontal) | |
| 5 | Knee, upright, side | Second | Stand | C For stand |

The “A evacuation position” in Table 8 refers to a position where the second bulb is to be evacuated when the X-ray tube 23 (first bulb) is used such that the second bulb is not brought into contact with the first bulb. In a similar manner to FIG. 6, the area surrounded by a thick frame in FIG. 7 is the movable range of the second bulb. Therefore, the “A evacuation position” is not particularly limited if the A evacuation position is within the range AREA, which is the movable range of the second bulb, and if the A evacuation position is a position other than the fluoroscopic table 21, the strut 22, and the first bulb of the fluoroscopic table unit 2. In FIG. 7, the coordinates of the “A evacuation position” are (100, 250).

Meanwhile, “skyline photography” in Table 8 refers to a technique to capture a kneecap by using the second bulb (X-ray tube 32) at an inclination angle θ (for example, about 15°) in a state where the subject M (patient) is sitting on a top plate (here, the fluoroscopic table 21 in a horizontal state) as shown in FIG. 8. As the FPD 7 in FIG. 8, a portable wireless FPD is used. The patient holds the FPD 7 on the femoral side of the knee, and the FPD 7 is placed slightly inclined on the knee side such that the kneecap (see the oval frame in FIG. 8) is obtained.

In order to simplify the calculation of the position of the second bulb, each position is expressed on two-dimensional coordinates when the inspection room is viewed from above as shown in FIG. 7 in a similar manner to FIG. 6. Each position is determined when the apparatus is installed. Table 9 summarizes a relationship of coordinates at each position.

TABLE 9

| | Position of second bulb | x | y |
|---|---|---|---|
| A | Evacuation position | 100 | 250 |
| B | For skyline | 300 | 100 |
| C | For stand | 200 | 150 |

At this time, the distances between respective positions can be calculated as shown in Table 10. In order to simplify the calculation, the moving cost is calculated by rounding the distance to the first digit in a similar manner to Table 3.

TABLE 10

| Path | Distance | Cost |
|---|---|---|
| A<=>B | 250 | 250 |
| A<=>C | 141.4213562 | 140 |
| B<=>C | 111.8033989 | 110 |

In Table 8, the second bulb starts moving from the “A evacuation position.” The total moving cost of the protocol list in the initial state as shown in Table 8 is as shown in Table 11.

TABLE 11

| | | Total moving |
|---|---|---|
| Path | Calculation of moving cost | cost |
| AACBAC | “0 + 140 + 110 + 250 + 140” | 640 |

A sequence of the protocol list that minimizes the total moving cost is considered. First, it is assumed that the protocols with the same position of the second bulb are always performed continuously. In this example, it is assumed that, in Table 8, the protocols at the position A of the second bulb of the protocol number 1 and the protocol number 4, and the protocols at the position C of the second bulb of the protocol number 2 and the protocol number 5 are always performed continuously. Possible combinations are 3. (3 factorial)=6 ways, which is the number of sorting at 3 locations A, B, and C. Table 12 shows the total moving cost for all the combinations.

TABLE 12

| | | Total moving |
|---|---|---|
| Path | Calculation of moving cost | cost |
| AABC | “0 + 250 + 110” | 360 |
| AACB | “0 + 140 + 110” | 250 |
| ABAC | “250 + 250 + 140” | 640 |
| ABCA | “250 + 110 + 140” | 500 |
| ACAB | “140 + 140 + 250” | 530 |
| ACBA | “140 + 110 + 250” | 500 |

From Table 12, the path with the smallest total moving cost is “AACB”, and the protocol list that is sorted in this sequence is as shown in Table 13.

TABLE 13

| | | | Position of | Position of |
|---|---|---|---|---|
| No | Protocol name | Bulb | patient | second bulb |
| 1 | Knee, decubitus, | First | Fluoroscopic | A Evacuation |
| | front | | table | position |
| | | | (horizontal) | |
| 4 | Knee, decubitus, | First | Fluoroscopic | A Evacuation |
| | side | | table | position |
| | | | (horizontal) | |
| 2 | Knee, upright, | Second | Stand | C For stand |
| | front | | | |
| 5 | Knee, upright, side | Second | Stand | C For stand |
| 3 | Knee, skyline | Second | Fluoroscopic | B For skyline |
| | photography | | table | |
| | | | (horizontal) | |

The initial state of the second bulb is “A evacuation position” as shown in the protocol list shown in Table 8, but the initial position of each component is not uniquely determined in practice. The initial position can be an arbitrary position depending on, for example, details of an immediately preceding inspection. Therefore, in this case, in step S20 (sort by apparatus moving distance priority), steps S21 and S22 shown in the flowchart of FIG. 3 (b) are performed.

### (Step S21) Input the Position at the Time of Sorting

The position detector attached to each component (position detector 33 shown in FIG. 2 when detecting the position of the second bulb) dynamically obtains the position at the time of sorting. Then, the position of the component detected by the position detector is transmitted and input to the input-output port 57 (see FIG. 2). Note that it is not always necessary to input the position detected by the position detector. The user may manually input the position of the component by using the input unit 54 of the control unit 5 (see FIG. 2) such as a pointing device or a button.

Furthermore, manual input and calculation input may be combined. For example, a case where the position of the FPD or a holding mechanism that holds the FPD has been registered by the photographing protocol and known will be described. The position of the X-ray tube may be calculated and input from the position of the FPD or the holding mechanism that holds the FPD registered by the photographing protocol, or from a value of a distance of a perpendicular line from a focal position of the X-ray tube to a detection surface of the FPD by the photographing protocol (SID: source to image receptor distance).

When the component is the second bulb and the position of the second bulb at the time of sorting is input, Table 14 summarizes a relationship of coordinates at each position.

TABLE 14

| | Position of second bulb | x | y |
|---|---|---|---|
| A | Evacuation position | 100 | 250 |
| B | For skyline | 300 | 100 |
| C | For stand | 200 | 150 |
| S | Position at time of sorting | 250 | 100 |

In Table 14, “S position at the time of sorting” indicates the position of the second bulb at the time of sorting. In Table 14, the coordinates of “S position at the time of sorting” are (250, 100).

At this time, the distances between respective positions can be calculated as shown in Table 15. In order to simplify the calculation, the moving cost is calculated by rounding the distance to the first digit in a similar manner to Table 3 and Table 10. This step S21 corresponds to the position input step in the present invention.

TABLE 15

| Path | Distance | Cost |
|---|---|---|
| A<=>B | 250 | 250 |
| A<=>C | 141.4213562 | 140 |
| A<=>S | 212.1320344 | 210 |
| B<=>C | 111.8033989 | 110 |
| B<=>S | 50 | 50 |
| C<=>S | 70.71067812 | 70 |

### (Step S22) Update Sorting

In Table 14, the second bulb starts moving from the “S position at the time of sorting.” In other words, update is performed to sort the photographing sequence with the input “S position at the time of sorting” of the second bulb as a starting point.

A sequence of the protocol list that minimizes the total moving cost is considered. In a similar manner to a case where the initial state of the second bulb is “A evacuation position”, the protocols with the same position of the second bulb are always performed continuously. In this example, it is assumed that, in Table 8, the protocols at the position A of the second bulb of the protocol number 1 and the protocol number 4, and the protocols at the position C of the second bulb of the protocol number 2 and the protocol number 5 are always performed continuously. Considering the “S position at the time of sorting” of the second bulb as a starting point, possible combinations are 3. (3 factorial)=6 ways, which is the number of sorting at 3 locations A, B, and C. Table 16 shows the total moving cost for all the combinations.

TABLE 16

| | | Total moving |
|---|---|---|
| Path | Calculation of moving cost | cos |
| SABC | “210 + 250 + 110” | 570 |
| SACB | “210 + 140 + 110” | 460 |
| SBAC | “50 + 250 + 140” | 440 |
| SBCA | “50 + 110 + 140” | 300 |
| SCAB | “70 + 140 + 250” | 460 |
| SCBA | “70 + 110 + 250” | 430 |

From Table 16, the path with the smallest total moving cost is “SBCA”, and the protocol list that is sorted in this sequence is as shown in Table 17. This step S22 corresponds to the photographing sequence update step in the present invention.

TABLE 17

| | | | Position of | Position of |
|---|---|---|---|---|
| No | Protocol name | Bulb | patient | second bulb |
| | (Details of | Second | (Position of | S Position at |
| | immediately | | patient in | time of |
| | preceding | | immediately | sorting |
| | inspection) | | preceding | |
| | | | inspection) | |
| 3 | Knee, skyline | Second | Fluoroscopic | B For skyline |
| | capturing | | table | |
| | | | (horizontal) | |
| 2 | Knee, upright, | Second | Stand | C For stand |
| | front | | | |
| 5 | Knee, upright, side | Second | Stand | C For stand |
| 1 | Knee, decubitus, | First | Fluoroscopic | A Evacuation |
| | front | | table | position |
| | | | (horizontal) | |
| 4 | Knee, decubitus, | First | Fluoroscopic | A Evacuation |
| | side | | table | position |
| | | | (horizontal) | |

### (Step S30) Sort by Setting Parameter

Returning to the flowchart of FIG. 3 (a), the following description is made. When the “Sort by setting parameter” button mc is selected, the photographing sequence is determined as follows. The photographing sequence is determined in ascending or descending order of the setting parameter values. A specific example will be described later.

The above steps S10, S20, and S30 correspond to the photographing sequence determining unit in the present invention.

The display aspect of the protocol list that has been sorted as described above may be either FIG. 9 (a) or FIG. 9 (b). For example, a case where the protocol list of the initial state as shown in Table 8 is sorted as shown in Table 17 with the position at the time of sorting as a starting point will be described. In the protocol list of the initial state as shown in Table 8, the protocol names are arranged in a sequence of “knee, decubitus, front”, “knee, upright, front”, “knee, skyline photography”, “knee, decubitus, side”, and “knee, upright, side.” In the protocol list where sorting is performed as shown in Table 17, the protocol names are arranged in a sequence of “knee, skyline photography”, “knee, upright, front”, “knee, upright, side”, “knee, decubitus, front”, and “knee, decubitus, side.”

In such an example, in a sequence in which photographing is performed as shown in FIG. 9 (a), “knee, skyline photography”, “knee, upright, front”, “knee, upright, side”, “knee, decubitus, front”, and “knee, decubitus, side” may be displayed sequentially from the top. Alternatively, as shown in FIG. 9 (b), each protocol may be displayed with a number of the sequence to perform assigned in the sequence of the initial state in Table 8.

According to the photographing sequence determination supporting method according to the present embodiment, the elements that determine the photographing sequence include at least a path that minimizes the moving distance or the moving time of the subject (“sort by patient moving distance priority” button ma in FIG. 4), and a path that minimizes the moving distance or the moving time of the component constituting the X-ray radiographic apparatus (“sort by apparatus moving distance priority” button mb in FIG. 4). In step S1 (select each button), one of respective elements (three buttons ma to mc in FIG. 4) is selected, and according to the selected element, the photographing sequence is determined in any one of steps S10, S20, and S30. That is, a user selects an element that matches a situation and determines the photographing sequence according to the selected element, thereby allowing the user to freely sort the photographing sequence according to the situation.

When the user selects the path that minimizes the moving distance or the moving time of the subject (“sort by apparatus moving distance priority” button mb), the photographing sequence is determined along the path. When the user selects the path that minimizes the moving distance or the moving time of the component constituting the X-ray radiographic apparatus (“sort by apparatus moving distance priority” button mb), the photographing sequence is determined along the path. In either case of the subject or the component, by allowing the user to select the path that minimizes the moving distance or the moving time and determining the photographing sequence along the selected path, the photographing sequence can be optimized automatically.

The photographing sequence determination supporting method according to the present embodiment preferably includes the position input step of inputting the position of the component (step S21: input the position at the time of sorting), and the photographing sequence update step of performing update to sort the photographing sequence with the input position of the component as a starting point (step S22: update sorting). When the user selects the path that minimizes the moving distance or the moving time of the component (“sort by apparatus moving distance priority” button mb), the shortest path changes with the current position of the component as a starting point. Therefore, by performing the position input step (step S21: input the position at the time of sorting) and the photographing sequence update step (step S22: update sorting), updating to sort the photographing sequence can be performed automatically with the input position of the component as a starting point.

Unless the component is moved between the previous inspection and the current inspection, the final position of the component in the previous inspection matches the current position of the component in the current inspection. Anyway, the final position of the component in the previous inspection may be input, or the current position of the component in the current inspection may be input. An input unit to be used in the position input step is not limited to a pointing device or a button manually input by the user, but may be an input port (input-output port 57 of FIG. 2 in the present embodiment). When the input unit is the input port (input-output port 57), a position detector that detects the position of the component is provided, and the position of the component detected by the position detector is transmitted to the input port (input-output port 57) and input.

In either case of the subject or the component, an algorithm to be used for searching for the path that minimizes the moving distance or the moving time is not particularly limited. For example, it is possible to use an algorithm such as a method of searching for the shortest path when all of a plurality of points A, B, . . . are visited like a picture drawn with one stroke (traveling salesperson problem).

In either case of the subject or the component, the photographing sequence determination supporting method according to the present invention preferably includes: a cost correction step of correcting the cost including the moving distance or the moving time in part of the path (cost correction using the moving cost correction screen shown in FIG. 5). Changing the moving distance or the moving time in part of the path without changing the path itself means “correcting the cost (distance or time)” as described also in the field of “Solution to Problem.” For example, in a case where the subject (patient) has a disease in the foot as described above, when photographing that includes getting on and off a table is included in a series of X-ray radiographic processes, even if the moving distance or the moving time is short, correction is made to increase the cost if getting on and off a table is included. In this way, the path that minimizes the moving distance or the moving time is searched for, taking into account the cost corrected according to the situation.

Another example of the elements other than the path that minimizes the moving distance or the moving time (“sort by setting parameter” button mc in FIG. 4) is a preset setting value. The photographing sequence is determined in the photographing sequence determination step (step S30: sort by setting parameter) in ascending order or descending order of the setting value. By using one of the various setting values (setting parameters) in photographing, such as the X-ray tube (bulb) to use or the X-ray detector to use, the photographing sequence is determined in ascending/descending order. For example, when there is a plurality of X-ray tubes or there is a plurality of X-ray detectors, the priority to use is set as the setting value, and photographing regarding the X-ray tube/X-ray detector with high priority is first performed sequentially.

For example, as described in the field of “Solution to Problem”, a case will be described in which in a system in which two “X-ray detectors to use” use battery-powered wireless FPDs, the battery of one “FPD 1” is dead and the power is not turned on. The setting value indicating priority of the other “FPD 2” is set at “1”, and the setting value indicating priority of “FPD 1” with the dead battery and the power turned off is set at “2.” It is possible to make settings to postpone the photographing using “FPD 1” in order to charge “FPD 1” and perform the inspection first by photographing using “FPD 2.” Remaining power of each wireless FPD may be monitored, and monitoring results (remaining power) may be converted into a numerical form and set as a setting value indicating priority. In this way, if the element is a preset setting value, this also produces an effect of being able to perform photographing without changing the photographing sequence of desired minimum photographing, regardless of other elements.

The photographing sequence determination supporting program according to the present embodiment (stored in the program memory 53 of FIG. 2) causes (the controller 56 of FIG. 2 in the present embodiment of a computer to execute the photographing sequence determination supporting method according to the present embodiment, thereby allowing the user to freely sort the photographing sequence according to the situation.

The X-ray radiographic apparatus 1 according to the present embodiment (see FIGS. 1 and 2) includes the arithmetic unit configured to execute the photographing sequence determination supporting program according to the present embodiment (controller 56 shown in FIG. 2 in the present embodiment), thereby allowing the user to freely sort the photographing sequence according to the situation.

The present invention is not limited to the above embodiment, but can be modified as follows.

(1) In the above embodiment, the X-ray radiographic apparatus 1 has a configuration shown in FIGS. 1 and 2, but is not limited to the configuration shown in FIGS. 1 and 2. For example, instead of the fluoroscopic table unit 2 including the fluoroscopic table 21, the strut 22, the X-ray tube 23, and the FPD 24 shown in FIG. 1, a fluoroscopic table unit including only the fluoroscopic table and the FPD without the strut or the X-ray tube may be provided, the ceiling traveling unit and the stand unit may be provided, and the X-ray tube of the ceiling traveling unit may also perform X-ray photography in the fluoroscopic table unit. A plurality of the X-ray tubes of the ceiling traveling unit may be provided. In addition, the present invention may be applied to an X-ray radiographic apparatus including a surgical photographing system and a support mechanism that supports the surgical photographing system (for example, a C arm), or an apparatus that incorporates an X-ray radiographic apparatus including a hand cart for visiting patients. The present invention is useful for an X-ray radiographic apparatus including a plurality of photographing systems.

(2) In the above embodiment, the digital X-ray detection unit such as the flat panel X-ray detector (FPD) is used as the X-ray detection unit. Meanwhile, an analog X-ray detection unit such as an image intensifier (I.I.) or an X-ray film may be used, or both the digital X-ray detection unit and the analog X-ray detection unit may be combined.

(3) In the above embodiment, the path that minimizes the moving distance of the subject or the moving distance of the component has been described. Instead of the moving distance, the present invention may be applied to a path that minimizes the moving time. In particular, when the moving speed of the component is not constant (for example, when the up-and-down speed of the component is slower than the horizontal moving speed), the present invention may be applied to a path that minimizes the moving time.

(4) In the above embodiment, as shown in FIGS. 6 and 7, the path that minimizes the moving distance or the moving time of the subject and the moving distance or the moving time of the component has been a path along a horizontal plane. However, this may be a path along an up-and-down direction, or the path along the horizontal plane and the path along the up-and-down direction may be combined.

(5) In the above embodiment, cost correction is performed for the subject (patient), but may be performed for the component. For example, when the component (for example, an X-ray tube or a fluoroscopic table) moves up and down, the cost may be corrected to increase.

### REFERENCE SIGNS LIST

1 X-ray radiographic apparatus
2 fluoroscopic table unit
23, 32 X-ray tube
3 ceiling traveling unit
4 stand unit
5 control unit
52 image memory unit
56 controller
7, 24, 42 flat panel X-ray detector (FPD)
ma “sort by patient moving distance priority” button
mb “sort by apparatus moving distance priority” button
mc “sort by setting parameter” button
M subject

1. A photographing sequence determination supporting method for supporting determination of a photographing sequence in a series of X-ray radiographic processes in association with movement of a subject or an X-ray radiographic apparatus, the photographing sequence determination supporting method comprising:
an element selection step of selecting one from elements that determine the photographing sequence, the elements including at least a path that minimizes a moving distance or a moving time of the subject, and a path that minimizes a moving distance or a moving time of a component constituting the X-ray radiographic apparatus; and
a photographing sequence determination step of determining the photographing sequence according to a selected element.

2. The photographing sequence determination supporting method according to claim 1, further comprising:
a position input step of inputting a position of the component; and
a photographing sequence update step of performing update to sort the photographing sequence with the input position of the component as a starting point.

3. The photographing sequence determination supporting method according to claim 1, further comprising a cost correction step of correcting a cost including the moving distance or the moving time in part of the path.

4. The photographing sequence determination supporting method according to claim 1, wherein
the elements are preset setting values, and
the photographing sequence is determined in the photographing sequence determination step in ascending order or descending order of the setting values.

5-6. (canceled)
